(19) United States
(12) Patent Application Publication
(10) Pub. No.: US 2007/0282243 A1
(43) Pub. Date: Dec. 6, 2007

(54) Apparatus for performing myocardial revascularization in a beating heart and left ventricular assistance condition

(75) Inventors: Patrizia Pini, Novedrate-Como (IT); Eugenio Cremascoli, Novedrate-Como (IT); Paolo Pepino, Novedrate-Como (IT)

Correspondence Address: HEDMAN & COSTIGAN P.C., 1185 AVENUE OF THE AMERICAS, NEW YORK, NY, 10036, US

(73) Assignee: N.G.C MEDICAL S.p.A

(21) Appl. No.: 11/545,869

(22) Filed: Oct. 11, 2006

(30) Foreign Application Priority Data
Jun. 1, 2006 (IT) MI2006A 001080

(51) Int. Cl. A61M 5/00; A61M 37/00

(52) U.S. Cl. 604/6.14; 604/8

(57) ABSTRACT

An apparatus for performing myocardial revascularization in a beating heart and left ventricular assistance condition comprises an arterial cannula, of a per se known type, arranged in the descending aorta, a novel arterial draining cannula, of a two cannula stage type, so arranged that the draining occurs from the left auricle and left ventricle, and a centrifugal pump allowing blood flow from the left ventricle to the aorta. The apparatus allow the patients to be operated on with the safety of a conventional extra-corporeal circulation and with a risk and cost reduction due to the off-pump technique.

### BACKGROUND OF THE INVENTION

The present invention relates to an apparatus for performing myocardial revascularization in a beating heart and left ventricular assistance condition.

Up to now, new myocardial revascularization procedures have been performed according to three operation modes, that is: by the conventional extracorporeal circulation technique (CEC), the reduced extracorporeal circulation technique (mini CEC), or the beating heart without extracorporeal circulation technique.

As known, extracorporeal circulation provides to exclude heart and lungs from the patient circulation system, to provide a stationary and bloodless operation field to be operated on by the heart surgeon.

In particular, the heart pumping function is provided by an outer pump, whereas the lung oxygenating and purifying function is provided by an outer oxygenating device.

The patient venous blood is drained from the right heart through suitable draining cannulas, and then being conveyed, through a conveying circuit, toward the oxygenating device and pump.

Hence, the oxygenated and purified blood follows the blood circuit toward a suitable aorta cannula, to be reintroduced into the patient.

The heart-lung machine, the used cannulas and blood circuit arrangement are performed in several embodiments, according to preferred hospital procedures.

Hereinbelow there will be briefly disclosed several arrangements of the blood circuit devices or components, for illustrative but not limitative purposes.

The venous cannula comprises a plastic tubular element perforated at set regions thereof, and is arranged in the right ventricle and/or top cava and/or bottom cava, to draining venous blood.

The venous blood is usually collected in a graduated reservoir, arranged upstream of the oxygenating device.

The oxygenating device generally comprises a cartridge including a plurality of polypropylene hollow fibers, therethrough an oxygenating gas mixture is conveyed; about said fibers venous blood is caused to flow in a counter-current relationship, thereby performing the desired gas exchanges with the gas mixture.

The pump is of a “roller” type, and, more specifically, conventionally comprise a two-roller system, pressing the tube therethrough blood flows, to apply a pushing force to the flowing blood.

On the other hand, the pump can also be of a “centrifugal” type, in which blood is urged by contoured helical members, turning under the effect of a magnetic field and held in a low-filling in chamber.

The arterial cannula, in turn, is introduced downstream of the aorta clamp, and comprise a plastic tubular element having an open tip, and a suitable size.

The most diffused risks in such a surgical operation comprise encephalopathy, neurocognitive problems and a systemic inflammatory response syndrome, as well as respiratory insufficiency.

The provision of the venous reservoir in the CEC circuit provides a good haemodilution of the patient blood.

In general, such a dilution is of 1.500 cc.

The above mentioned haemodilution requires that the patients be subjected to substantial blood transfusions.

To limit haemodilution in patients subjected to an extracorporeal circulation, a technique has been developed in recent years, providing to eliminate the reservoir and arrange the oxygenating device directly on the blood circuit venous line.

This technique is the so-called closed perfusion system.

Recent searches have demonstrated that this technique or procedure brings about a less inflammatory response by the patient.

The beating heart or “off pump” technique, on the other hand, is performed without using the above disclosed circuit.

By this technique, the patient is operated on while holding operating his/her vital functions: the heart surgeon, accordingly, operates on a beating heart with the patient lungs performing the gaseous exchanges.

The stabilization of the operation situ, i.e. of the coronary artery being revascularized, is achieved by using suitable stabilizing elements which “stretch” the operation area by applying a localized pressure, either positive or negative, on the myocardium.

The advantages of an off-pump operation are a less hospitalization time, a less auricle fibrillation, a less inflammatory response, a less involvement of other body members, and a less post-operative bleeding.

Yet other advantages are of an economic type such as a less requirement of medical devices or tools and a less patient hospitalization type.

The disadvantages are related, sometime, to a not completely full revascularization, if the heart surgeon has not a full skillness in beating heart coronary surgical operations.

In fact, this technique requires a good manual handling by the heart surgeon, and a proper managing or monitoring of the heart pressure parameters.

This technique or procedure is rather dangerous for patient having a low election fraction, with a critical stenosis of the left coronary artery common trunk, with unstable angina and calcific or intramyocardic coronary arteries.

These patients, in particular, are very sensible to short haemodynamic variations.

Because of the above mentioned reasons, an initially off-pump surgical procedure is frequently converted to an on-pump procedure.

Recent searched have demonstrated that an off to on pump reversal greatly worsens mortality and morbidity risks in affected patients.

### SUMMARY OF THE INVENTION

Accordingly, the aim of the present invention is to provide an apparatus allowing to carry out a novel myocardial revascularization operating technique, to overcome the above drawbacks affecting the prior art.

Within the scope of the above mentioned aim, a main object of the invention is to provide such an apparatus allowing to perform a myocardial revascularization in a beating heart, with a left ventricular assistance.

Another object of the invention is to provide such an apparatus allowing high risk patients to be operated, i.e. hold or haemodynamically unstable patients, with a low election fraction, which cannot be percutaneously processed (by angioplastic methods or stent implantation methods) and which patients moreover are at risk during an on and off pump operation, with the same safety as a conventional extracorporeal circulation method, and with the great reductions of the risks and expenses associated to the off-pump technique.

According to one aspect of the present invention, the above mentioned aim and objects, as well as yet other objects, which will become more apparent hereinafter, are achieved by an apparatus for performing myocardial revascularization in a beating heart and left ventricular assistance condition, characterized in that said apparatus comprises an arterial cannula, arranged in a descending aorta, a two-stage arterial draining cannula, so arranged that draining occurs from a left auricle and left ventricle, and a centrifugal pump allowing flow from the left ventricle to the aorta.

### BRIEF DESCRIPTION OF THE DRAWINGS

Further characteristics and advantages of the present invention will become more apparent hereinafter from the following detailed disclosure of a preferred, though not exclusive, embodiment of the invention which is illustrated, by way of an indicative, but not limitative, example in the accompanying drawings, where:

FIG. 1 is a perspective view schematically showing an application of the apparatus according to the present invention;

FIG. 2 is a side view of the cannula included in the apparatus according to the present invention;

FIG. 3 is a cross-sectional view showing the cannula;

FIG. 4 is a further side view, on an enlarged scale, of a middle portion of the cannula;

FIG. 5 is a further side view, on an enlarged scale, of the cannula tip portion; and

FIG. 6 is a further side view, on an enlarged scale, of a middle portion of the cannula shown in FIG. 2, according to a different arrangement thereof.

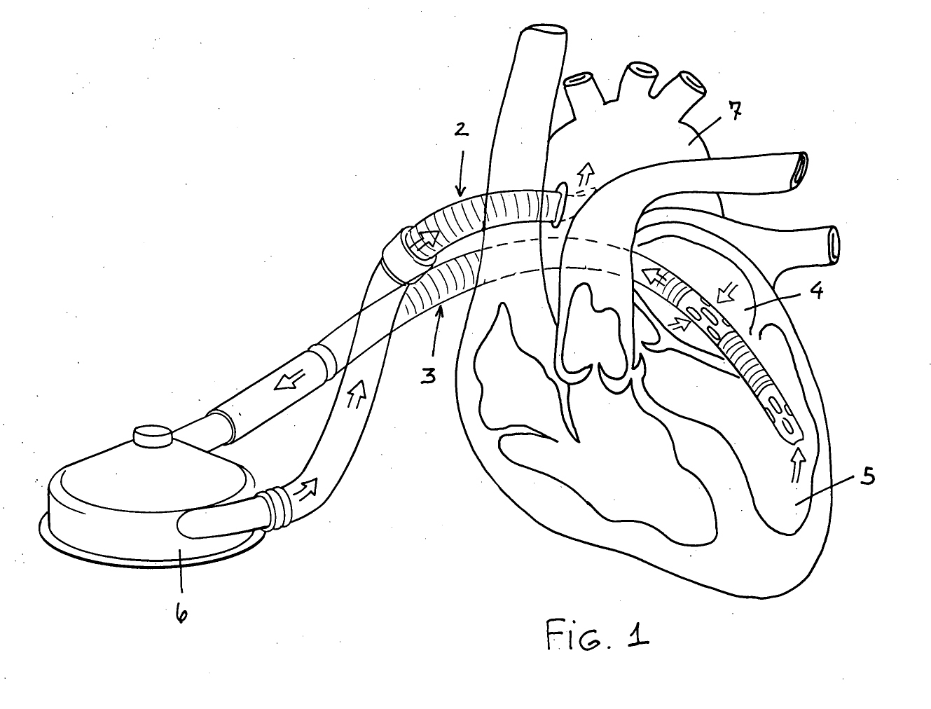

### DESCRIPTION OF THE PREFERRED EMBODIMENTS

With reference to the number references of the above mentioned figures, the apparatus according to the invention, which has been generally indicated by the reference number 1, comprises an arterial cannula 2, of a per se known type, arranged in the descending aorta, a novel arterial draining cannula 3, of a two-stage type, so arranged that draining occurs from the left auricle 4 and left ventricle 5, and a centrifugal pump 6 allowing blood to flow from the left ventricle 5 to the aorta 7.

The absence of the oxygenating device and heat exchanger, in addition to the possibility of arranging the centrifugal pump near the head of the patient, reduces to about 120 cc the filling-in of the circuit.

The reservoir of the circuit, preferably in the form of a bag with a 20-mycron filter, only operates to fill-in the circuit and has a recirculation means.

The operation is performed as a conventional beating heart surgical operation.

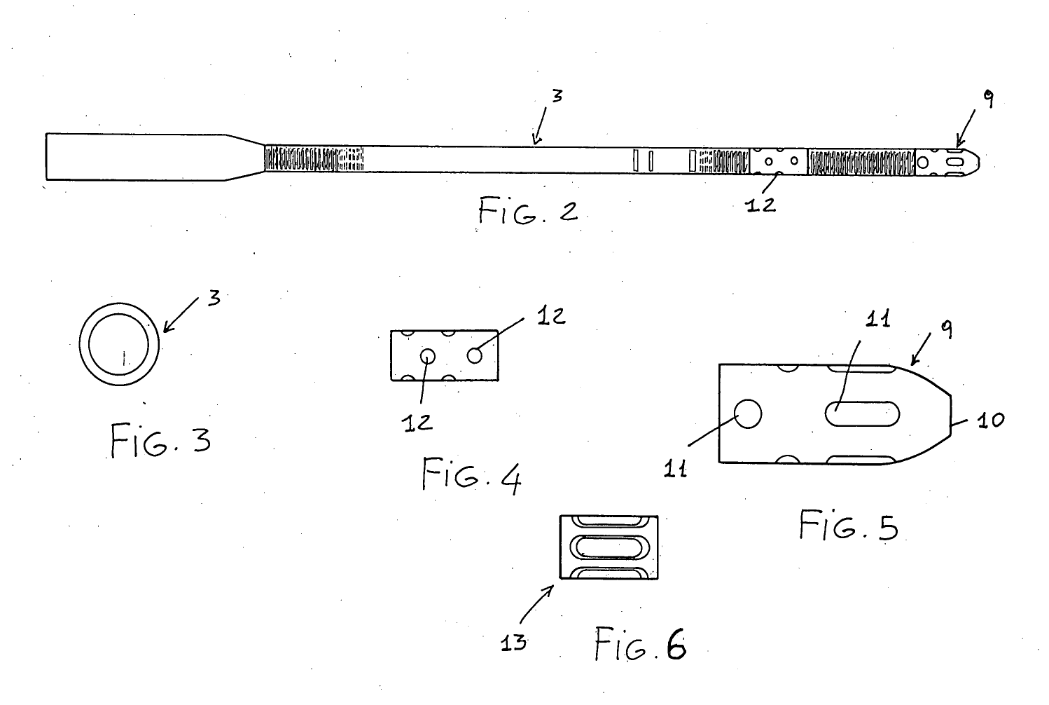

The novel cannula for draining arterial blood from the left heart, which cannula has been generally indicated by the reference number 3, is shown in FIG. 2.

More specifically, the cannula 3 comprises a suitably perforated tubular element, therethrough arterial blood is urged to the aorta through the above disclosed blood circuit.

In particular, said cannula 3 is preferably made of a PVC or polyurethane material, and, preferably, is of a type stiffened by a metal coil 8, for example an AISI304 metal coil, thereby it cannot be bent during the use, with a consequence of locking or shutting off the blood flow, and thereby preventing the so-called kinking phenomenon.

The cannula 3 can also comprise a shutter-spindle element, of a malleable type, facilitating the engaging of the cannula, and preventing, in such an operating step, the blood from flowing outward.

The provision of the metal coil element 8, the called armoring element, allows to provide a soft and atraumatic material cannula, of thin construction and adapted to resist against kinking.

The small thickness of the cannula 3 will further reduce its size, thereby providing a clear advantage, with respect to an operation through the circulatory system.

The tip portion 9 of the cannula is of an open type and, more preferably, of a bullet tip type, with a broad or enlarged hole 10 at the tip portion thereof.

Said tip portion can also be obtained by molding and glued to the cannula body.

The curvet arrangement of the tip allows the cannula to be easily introduced into the blood vessel, and the broad hole 10 thereof will provide a comparatively high draining.

Further side holes 11 for providing a very great draining capability are moreover provided.

The above disclosed cannula cross section arrangement has been specifically designed for engaging in a ventricle.

A second set of holes 12 are provided starting at about 30 mm from the tip holes.

Said holes 12 being designed for a left auricle draining.

This second perforated region 12 of the cannula, on the other hand, could be easily bent and, for overcoming such a drawback, it has been properly stiffened or reinforced.

In particular, the stiffening or reinforcement elements can comprise the spiral or coil element 8 thereabout the holes 12 are arranged, or a stiffen or rigid material basket element 13, embedded in the cannula material and thereon said holes are provided.

Said basket element can also be obtained by molding and glueing it to the cannula.

In the preferred embodiment, the cannula has the region of the holes 12 made of rigid polyurethane (ex. 95 Sh.A) and being embedded in the soft PVC or PU (ex. 65 Sh.A).

The draining holes 12 are just formed on this stiffening or reinforcement element or material.

Said holes 12 are preferably of a circular type and are arranged as a fishbone, since they, with such a configuration, do not weak the perforated region.

In this connection it should be apparent that said holes 12 could also be arranged along a spiral line or have an elliptical configuration.

The apparatus according to the present invention allows to easily perform a myocardial revascularization under a beating heart—left ventricular assistance condition.

As stated, this technique is very useful for high risk patients, that is old patients, or haemodynamically unstable or low election fraction patients, which cannot be percutaneously processed (by angioplastic or stent implantation methods) and which, in the meanwhile, are at least in an on and off pump surgical operation.

This technique allows to operate on said patients with the safety of a conventional extra-corporeal circulation operation, and with a great reduction of risks and costs owing to the off-pump technique.

In this connection it should be moreover pointed out that the left ventricle assistance technique is such as to easily drain oxygenated blood from the left ventricle, to reintroduce said oxygenated blood into the aorta by a pump, which pump may be arranged either outward or inward of the draining cannula.

In the latter case, the pump is a miniaturized turbo-pump.

The left ventricular assistance is used to support the earth pumping function, for example waiting for a transplantation operation.

The advantages afforded by the present invention are those associated with the beating heart technique, in which, however, the ventricular assistance provides a favorable decompression of the left ventricle, thereby facilitating the handling and stabilizing of the heart.

In particular, it is possible to also operate on patient having a very low ejection fraction, since the left ventricle is not stressed, to provide a haemodilution near to zero (of about 120 cc).

Thus, by using the technique or procedure according to the present invention, myocardial revascularization is performed on a beating heart, with an additional left ventricle assistance, performed through a small size extracorporeal circuit.

It has been found that the invention fully achieves the intended aim and objects.

In fact, the invention has provided an apparatus including a dedicated circuit and a specifically designed cannula, allowing to perform a novel myocardial revascularization operating technique.

On the contrary, commercially available prior cannulas are limitedly used for venous draining and are designed to be arranged in the right auricle and in the cava vena.

Thus, the apparatus according to the invention, and in particular of the novel draining cannula for providing draining from the left auricle and ventricle, allow to operate on the patients in a beating heart condition, with the above stated advantages, in particular for unstable and low ejection fraction patients.

The apparatus according to the invention, accordingly, allows to greatly reduce the risks and costs of cardiovascular by-pass, both in an on-pump and in an off-pump condition, thereby fully eliminating the possibility of switching an off-pump operation to an on-pump operation.

Moreover, the reduced priming requirements of the system allows to consequently reduce the use of anticoagulating substances and the risks of post-operating transfusions, which are typical of an on-pump operation.

Furthermore, the subject method is very simple and can be very easily carried out.

In practicing the invention, the used materials, together with the contingent size, can be any, depending on requirements.

In this connection it should be pointed out that the disclosed cannulas and/or pump and/or circuit are coated by coating materials designed for providing a high haemo-compatibility, for example heparin or synthetic derivatives thereof, such as phosphochlorine.

1. An apparatus for performing myocardial revascularization in a beating heart and left ventricular assistance condition, characterized in that said apparatus comprises an arterial cannula, arranged in a descending aorta, a two-stage arterial draining cannula, so arranged that draining occurs from a left auricle and left ventricle, and a centrifugal pump allowing flow from the left ventricle to the aorta.

2. An apparatus for performing myocardial revascularization in a beating heart and left ventricular assistance condition, according to claim 1, characterized in that said arterial draining cannula comprises a perforated tubular element made of a PVC or polyurethane material.

3. An apparatus for performing myocardial revascularization in a beating heart and left ventricular assistance condition, according to claim 1, characterized in that said arterial draining cannula comprises a stiffening element to make said cannula resistant against kinking.

4. An apparatus for performing myocardial revascularization in a beating heart and left ventricular assistance condition, according to claim 3, characterized in that said stiffening element for stiffening said arterial draining cannula comprises a metal coil made of an AISI304 steel material.

5. An apparatus for performing myocardial revascularization in a beating heart and left ventricular assistance condition, according to claim 1, characterized in that said cannula has an open cannula tip, of a bullet tip type and with a large tip hole.

6. An apparatus for performing myocardial revascularization in a beating heart and left ventricular assistance condition, according to claim 5, characterized in that said tip is so curved as to facilitate an engaging of said cannula in blood vessels, said cannula hole providing a high draining capability.

7. An apparatus for performing myocardial revascularization in a beating heart and left ventricular assistance condition, according to claim 5, characterized in that said draining cannula tip comprises a plurality of side holes designed for providing a maximum draining capability.

8. An apparatus for performing myocardial revascularization in a beating heart and left ventricular assistance condition, according to claim 5, characterized in that said draining cannula tip comprises a plurality of holes arranged starting at about 30 mm of the tip holes and provided for draining the left auricle.

9. An apparatus for performing myocardial revascularization in a beating heart and left ventricular assistance condition, according to claim 8, characterized in that said second plurality of holes are arranged at a cannula region including a stiffening or reinforcement spiral element thereabout said holes are arranged.

10. An apparatus for performing myocardial revascularization in a beating heart and left ventricular assistance condition, according to claim 9, characterized in that said region of said second hole plurality is made of a rigid polyurethane material (ex. 95 Sh.A) and embedded in a PVC or PU soft material (ex. 65 Sh.A)

11. An apparatus for performing myocardial revascularization in a beating heart and left ventricular assistance condition, according to claim 9, characterized in that said holes of said region of said second plurality of holes have a circular cross section and are arranged according to a fishbone arrangement, thereby preventing the perforated region from being weakened.

12. An apparatus for performing myocardial revascularization in a beating heart and left ventricular assistance condition, according to claim 9, characterized in that said second plurality of holes are arranged along a spiral line and have an elliptical configuration.

13. An apparatus for performing myocardial revascularization in a beating heart and left ventricular assistance condition, according to claim 9, characterized in that said second plurality of holes comprises a rigid material basket element embedded in the cannula material and thereon said holes are formed.

14. An apparatus for performing myocardial revascularization in a beating heart and left ventricular assistance condition, according to claim 13, characterized in that said rigid material basket element is glued to the other portions of said cannula.

15. An apparatus for performing myocardial revascularization in a beating heart and left ventricular assistance condition, according to claim 1, characterized in that said apparatus is adapted to perform a myocardial revascularization in a beating heart—left ventricular assistance condition.

16. An apparatus for performing myocardial revascularization in a beating heart and left ventricular assistance condition, according to claim 1, characterized in that, since said apparatus is lacking of an oxygenating device and a heat exchanger, said centrifugal pump can be arranged near a head of a patient, while reducing to about 120 cc the filling-in of the circuit.

17. An apparatus for performing myocardial revascularization in a beating heart and left ventricular assistance condition, according to claim 1, characterized in that said pump can be arranged either outside or inside said draining cannula, with the pump arranged inside said draining cannula, said pump comprising a miniaturized turbo-pump.

18. An apparatus for performing myocardial revascularization in a beating heart and left ventricular assistance condition, according to claim 1, characterized in that said apparatus is adapted to perform a myocardial revascularization on a beating art, with an additional left ventricular assistance, said left ventricular assistance being performed through a small extracorporeal circuit.

19. An apparatus for performing myocardial revascularization in a beating heart and left ventricular assistance condition, according to claim 1, characterized in that said apparatus comprises a reduced priming allowing to reduce an anti-coagulating material use and risks of post-operating transfusions typical in an on-pump surgical operation.

20. An apparatus for performing myocardial revascularization in a beating heart and left ventricular assistance condition, according to claim 1, characterized in that said cannulas and/or pumps and/or circuits included in said apparatus are coated by coating material providing a high haemo-compatibility, for example heparin or derivatives thereof, or of a synthetic origin, such as phosphor-chlorine.
